(19) United States
(12) Patent Application Publication
(10) Pub. No.: US 2018/0070986 A1
(43) Pub. Date: Mar. 15, 2018

(54) Pedicle Reconstruction Device

(71) Applicant: Spinal Balance, Inc., Toledo, OH (US)

(72) Inventors: Anand Agarwal, Ottawa Hills, OH (US); Adam MacMillan, Quincy, MA (US); Aakash Agarwal, Ottawa Hills, OH (US)

(73) Assignee: Spinal Balance, Inc., Toledo, OH (US)

(21) Appl. No.: 15/557,480

(22) PCT Filed: Mar. 14, 2016
(86) PCT No.: PCT/US2016/022307

§ 371 Date: Sep. 12, 2017

Related U.S. Application Data
(60) Provisional application No. 62/131,995, filed on Mar. 12, 2015.

(51) Int. Cl. A61B 17/68; A61B 17/70; A61B 17/86

(52) U.S. Cl. CPC A61B 17/686; A61B 17/8625; A61B 17/7061

(57) ABSTRACT

A pedicle reconstruction device adapted for use with a pedicle screw includes a head portion, a stem portion extending from the head portion, a neck portion extending from the stem portion, and an anchor portion extending from the neck portion. A slot is formed through the neck portion and the anchor portion such that the anchor portion can be oriented in a retracted condition when pedicle screw is not disposed therein and in an extended condition when a pedicle screw is disposed therein.

### CROSS-REFERENCE TO RELATED APPLICATIONS

This application claims the benefit of U.S. Provisional Application No. 62/131,995, filed Mar. 12, 2015, the disclosure of which is incorporated herein by reference.

### BACKGROUND OF THE INVENTION

This invention relates in general to the field of orthopedic surgical implants. In particular, this invention relates to an improved structure for a pedicle reconstruction device that provides strength to a pedicle in a vertebra of a spine and thereby facilitates the installation and use of a pedicle screw therewith.

A variety of spinal fixation devices are known for treating scoliosis, spondylolisthesis, degenerative disc disease, vertebra fractures, and other spinal disorders or abnormalities. In many instances, such spinal fixation devices include one or more pedicle screws, which are individually secured to the vertebrae of the spine to provide anchor points that can then be connected together with a rod or other alignment or immobilization structure. A typical pedicle screw includes a threaded shaft portion having a yoke-shaped head portion extending therefrom. The threaded shaft portion is adapted to be secured to a vertebra of the spine, while the head portion is adapted to be connected to the rod or other alignment or immobilization structure.

A vertebra of a spine includes a vertebra body, two pedicles, a spinal canal, a spinous process. The pedicles typically provide about 60% of the strength for any pedicle screw fixation done for lower back pain. In vertebrae experiencing osteopenia or osteoporosis, the density of the bone is decreased, which weakens the boney structure therein. When a pedicle screw is inserted into a vertebra, two problems may occur. First, misplacement of the pedicle screw during installation may damage the medial wall of the pedicle of the vertebra, making revision surgery difficult and screw fixation weak. This happens more often in weaker bones, but can also occur in normal bone. Second, the fixation of the pedicle screw may not be strong enough. This can result in undesirable loosening of the pedicle screw following installation. The incidence of pedicle screw misplacement reported rates range from about 10% to about 40%. Aging patients, who usually require spinal instrumentation as a treatment for instabilities or deformities, are of significant concern. Pedicle screws are the workhorse of spinal instrumentation for the adult spine, and screw pullout is a major concern in the osteoporotic patient and adult spinal deformity in the osteoporotic spine.

One known mechanism for improving screw fixation in osteoporotic vertebrae is often referred to as pedicle and vertebral body augmentation. Polymethyl methacrylate (PMMA) is typically used to inter-digitate with surrounding trabecular bone to increase fixation strength and firmly anchor the screw. PMMA screw augmentation increases the stability of the pedicle screw and, therefore, also increases the pull-out strength. To facilitate the use of PMMA, pedicle screws have been designed with fenestration in the distal one-third of the screw to inject cement therethrough to stabilize the screw around the fenestration. However, known methods of this type provide fixation only in the vertebral body, but not in the pedicles of the vertebra.

Pedicle screws rely primarily on cancellous bone for purchase, with the pedicle providing approximately 60% of the pullout strength. This invention, therefore, relates to a pedicle reconstruction device that provides strength to the pedicle of the vertebra that facilitates the installation and use of these pedicle screws.

### SUMMARY OF THE INVENTION

This invention relates to an improved structure for a pedicle reconstruction device that provides strength to a pedicle in a vertebra of a spine and thereby facilitates the installation and use of a pedicle screw therewith. The pedicle reconstruction device includes a head portion, a stem portion extending from the head portion, a neck portion extending from the stem portion, and an anchor portion extending from the neck portion. A slot is formed through the neck portion and the anchor portion such that the anchor portion can be oriented in a retracted condition when pedicle screw is not disposed therein and in an extended condition when a pedicle screw is disposed therein.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a plan view of a vertebra of a spine including a vertebral body, two pedicles, a spinous process, and a spinal canal, and further showing typical placements of two pedicle screws therein.

FIG. 2 is a plan view of the vertebra of the spine illustrated in FIG. 1 schematically showing typical placements of two pedicle reconstruction devices in accordance with this invention.

FIG. 3 is a side elevational view of a first embodiment of a pedicle reconstruction device in accordance with this invention, shown in a retracted condition.

FIG. 4 is a perspective view of the first embodiment of the pedicle reconstruction device shown in FIG. 3.

FIG. 5 is a sectional side elevational view of the first embodiment of the pedicle reconstruction device shown in FIGS. 3 and 4.

FIG. 6 is a sectional perspective view of the first embodiment of the pedicle reconstruction device shown in FIGS. 3 through 5.

FIG. 7 is an end elevational of the first embodiment of the pedicle reconstruction device shown in FIGS. 3 through 6.

FIG. 8 is a side elevational view of the first embodiment of the pedicle reconstruction device similar to FIG. 3, shown in an expanded condition.

FIG. 9 is a plan view of the typical vertebra of the spine illustrated in FIG. 2 having two of the first embodiment of the pedicle reconstruction devices provided therein, wherein both of the pedicle reconstruction devices are shown in the retracted condition.

FIG. 10 is a plan view of the typical vertebra of the spine similar to FIG. 9, wherein both of the pedicle reconstruction devices are both shown in the expanded condition.

FIG. 11 is a sectional perspective view of a portion of a second embodiment of a pedicle reconstruction device in accordance with this invention.

FIG. 12 is an enlarged perspective view of a portion of the second embodiment of the pedicle reconstruction device shown in FIG. 11.

FIG. 13 is a sectional perspective view of a portion of a third embodiment of the pedicle reconstruction device in accordance with this invention.

FIG. 14 is an enlarged perspective view of a portion of the third embodiment of the pedicle reconstruction device shown in FIG. 13.

FIG. 15 is a sectional elevational view of a fourth embodiment of a pedicle reconstruction device in accordance with this invention.

FIG. 16 is a sectional perspective view of the fourth embodiment of the pedicle reconstruction device shown in FIG. 15.

FIG. 17 is an end elevational view of the fourth embodiment of the pedicle reconstruction device shown in FIGS. 15 and 16.

FIG. 18 is a side elevational view of a fifth embodiment of a pedicle reconstruction device in accordance with this invention.

FIG. 19 is a perspective view of the fifth embodiment of the pedicle reconstruction device shown in FIG. 18.

FIG. 20 is a sectional perspective view of the fifth embodiment of the pedicle reconstruction device shown in FIGS. 18 and 19.

FIG. 21 is a side elevational view of a sixth embodiment of a pedicle reconstruction device in accordance with this invention.

FIG. 22 is a perspective view of the sixth embodiment of the pedicle reconstruction device shown in FIG. 21.

FIG. 23 is a sectional perspective view of the sixth embodiment of the pedicle reconstruction device shown in FIGS. 21 and 22.

FIG. 24 is a side elevational view of a seventh embodiment of a pedicle reconstruction device in accordance with this invention.

FIG. 25 is a perspective view of the seventh embodiment of the pedicle reconstruction device shown in FIG. 24.

FIG. 26 is a sectional perspective view of the seventh embodiment of the pedicle reconstruction device shown in FIGS. 24 and 25.

FIG. 27 is an exploded perspective view of an eighth embodiment of the pedicle reconstruction device in accordance with this invention.

FIG. 28 is an assembled perspective view of the eighth embodiment of the pedicle reconstruction device shown in FIG. 27, shown in a retracted condition.

FIG. 29 is an assembled view of the eighth embodiment of the pedicle reconstruction device shown in FIG. 27, shown in an expanded condition.

FIG. 30 is a perspective view of a ninth embodiment of a pedicle reconstruction device in accordance with this invention.

FIG. 31 is a sectional elevational view of the ninth embodiment of the pedicle reconstruction device shown in FIG. 30.

FIG. 32 is a perspective view of a tenth embodiment of a pedicle reconstruction device in accordance with this invention.

FIG. 33 is a sectional perspective view of the tenth embodiment of the pedicle reconstruction device shown in FIG. 32.

FIG. 34 is an assembled side elevational view of a conventional pedicle screw and the tenth embodiment of the pedicle reconstruction device shown in FIGS. 32 and 33.

FIG. 35 is an assembled sectional elevational view of the conventional pedicle screw and the tenth embodiment of the pedicle reconstruction device shown in FIG. 34.

FIG. 36 is a perspective view of an eleventh embodiment of a pedicle reconstruction device in accordance with this invention.

FIG. 37 is a sectional elevational view of the eleventh embodiment of the pedicle reconstruction device shown in FIG. 36.

FIG. 38 is a perspective view of a twelfth embodiment of a pedicle reconstruction device in accordance with this invention.

FIG. 39 is a perspective view of a thirteenth embodiment of a pedicle reconstruction device in accordance with this invention.

FIG. 40 is a perspective view of a fourteenth embodiment of a pedicle reconstruction device in accordance with this invention.

FIG. 41 is another perspective view of the fourteenth embodiment of the pedicle reconstruction device in accordance with this invention.

FIG. 42 is a perspective view of a fifteenth embodiment of a pedicle reconstruction device in accordance with this invention.

FIG. 43 is an exploded perspective view of a conventional pedicle screw and the fifteenth embodiment of the pedicle reconstruction device shown in FIG. 42.

FIG. 44 is an assembled perspective view of the conventional pedicle screw and the fifteenth embodiment of the pedicle reconstruction device shown in FIG. 43.

### DETAILED DESCRIPTION OF THE PREFERRED EMBODIMENTS

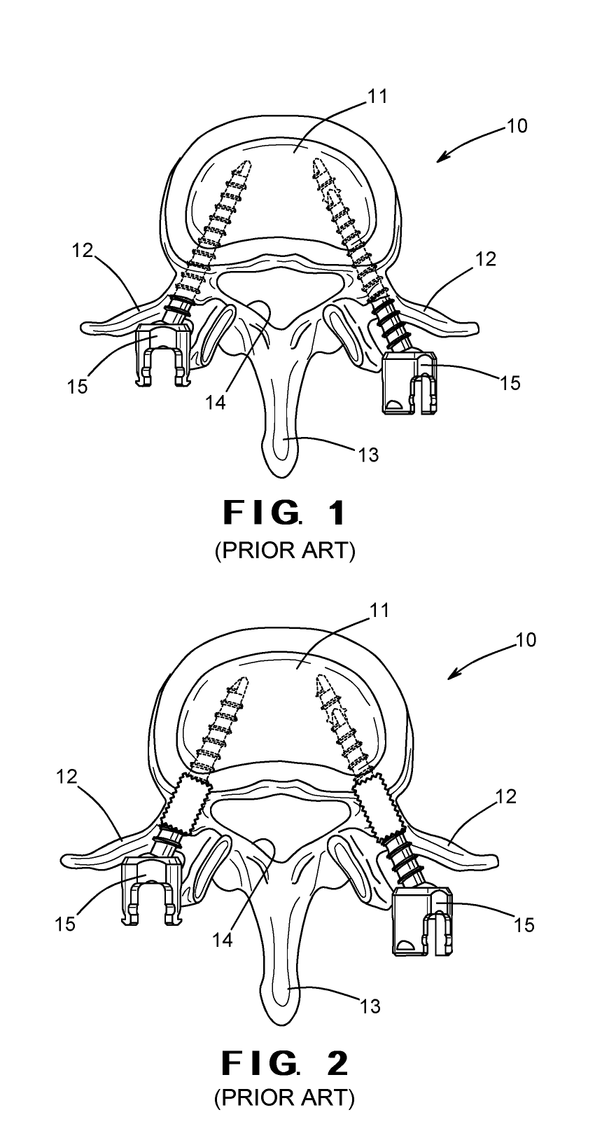

Referring now to the drawings, there is illustrated in FIGS. 1 and 2 a vertebra, indicated generally at 10, of a spine. As is well known, the vertebra 10 includes a vertebral body 11, two pedicles 12, a spinous process 13, and a spinal canal 14. This invention relates to an improved structure for a pedicle reconstruction device that provides strength to the pedicles 12 of the vertebra 10 and, therefore, facilitates the installation and use of one or more pedicle screw 15 therewith. FIG. 2 schematically illustrates typical placements of two pedicle reconstruction devices of the types described and illustrated herein, along with the associated conventional pedicle screws 15 installed in the two pedicles 12 of the vertebra 10. The specific structures and manners of operation of the various embodiments of the pedicle reconstruction devices of this invention will be described in detail below.

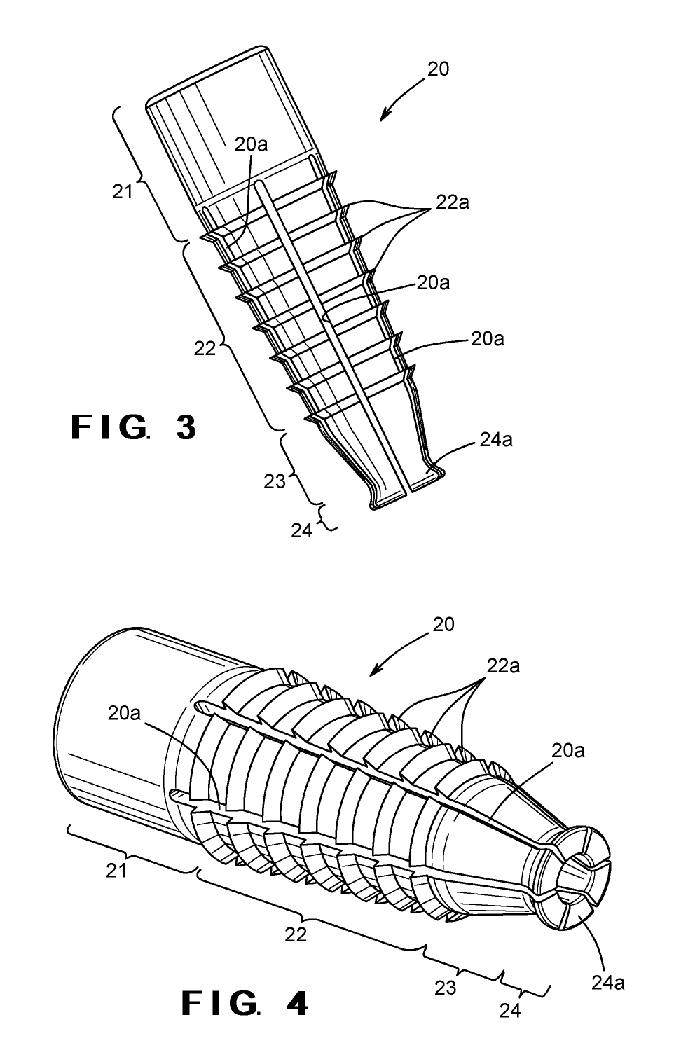

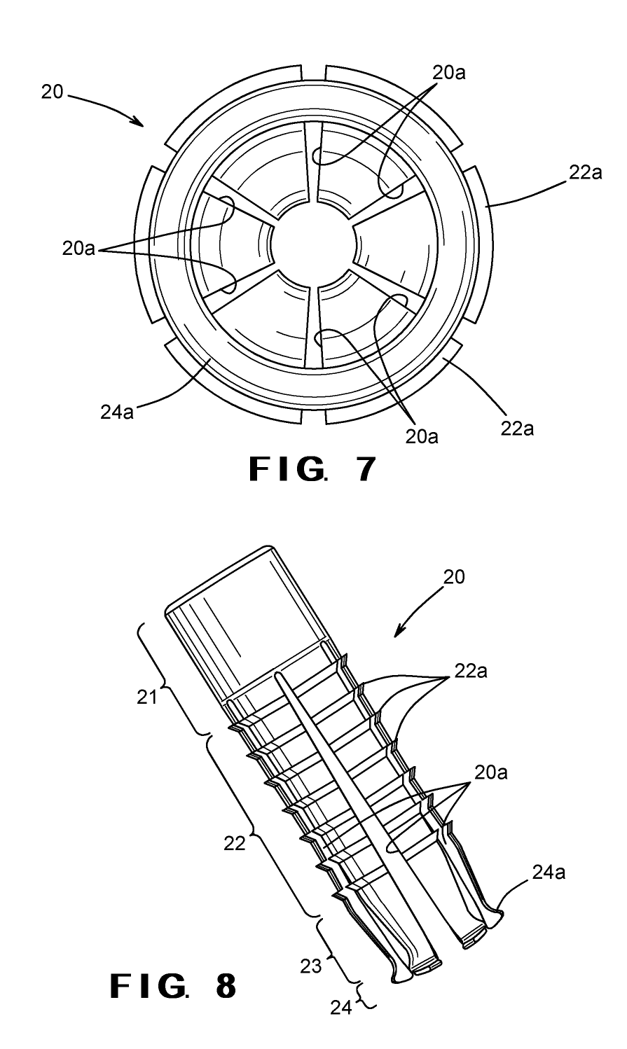

FIGS. 3 through 8 illustrate a first embodiment of a pedicle reconstruction device, indicated generally at 20, in accordance with this invention. FIGS. 3 through 7 show the first embodiment of the pedicle reconstruction device 20 in a retracted condition (i.e., prior to the installation of a pedicle screw 15 therein), while FIG. 8 shows the first embodiment of the pedicle reconstruction device 20 in an expanded condition (i.e., after the installation of a pedicle screw 15 therein). The first embodiment of the pedicle reconstruction device 20 is generally hollow and cylindrical in shape and includes a head portion 21, a stem portion 22, a neck portion 23, and an anchor portion 24.

The first embodiment of the pedicle reconstruction device 20 has a plurality of slots 20a provided therein. In the illustrated embodiment, six generally axially extending slots 20a are equidistantly provided about the circumference of the first embodiment of the pedicle reconstruction device 20. However, a greater or lesser number of such slots 20a (or none at all) may be provided in any desired locations. In the illustrated embodiment, each of the slots 20a extends from the head portion 21 completely through the stem portion 22, the neck portion 23, and the anchor portion 24. However, the slots 20a may extend through any desired portion or combinations of portions of the first embodiment of the pedicle reconstruction device 20 and may differ in length if desired. The slots 20a are provided to facilitate the expansion of the first embodiment of the pedicle reconstruction device 20 from the retracted condition (prior to the installation of a pedicle screw 15 therein) to the expanded condition (after the installation of a pedicle screw 15 therein), as will be explained in greater detail below.

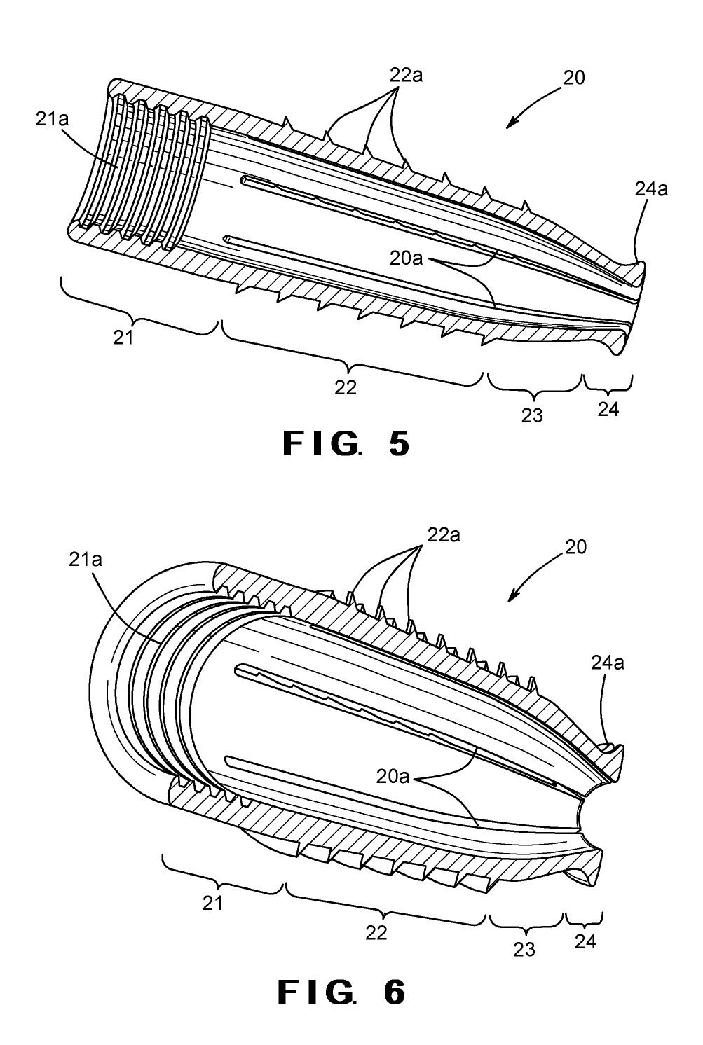

The outer surface of the head portion 21 of the first embodiment of the pedicle reconstruction device 20 is preferably smooth to facilitate the installation thereof into the pedicle 12 of the vertebra 10. However, the outer surface of the head portion 21 of the first embodiment of the pedicle reconstruction device 20 may have any desired shape or texture. Conversely, the inner surface of the head portion 21 of the first embodiment of the pedicle reconstruction device 20 is preferably serrated (such as shown at 21a in FIGS. 5 and 6) or is otherwise non-smooth in shape to facilitate the secure engagement of the pedicle screw 15 when inserted therethrough. However, the inner surface of the head portion 21 of the first embodiment of the pedicle reconstruction device 20 may have any desired shape or texture.

The outer surface of the stem portion 22 of the first embodiment of the pedicle reconstruction device 20 is preferably provided with a plurality of annular barbs (such as shown at 22a in FIGS. 3 through 8) or otherwise non-smooth shape to facilitate the secure engagement thereof with the pedicle 12 of the vertebra 10. However, the outer surface of the stem portion 22 of the first embodiment of the pedicle reconstruction device 20 may have any desired shape or texture. Conversely, the inner surface of the stem portion 22 of the first embodiment of the pedicle reconstruction device 20 is preferably smooth. However, the inner surface of the stem portion 22 of the first embodiment of the pedicle reconstruction device 20 may have any desired shape or texture.

The neck portion 23 of the first embodiment of the pedicle reconstruction device 20 preferably tapers from a relatively large size adjacent the stem portion 22 down to relatively small size adjacent the anchor portion 24, although such is not required. The anchor portion 24 of the first embodiment of the pedicle reconstruction device 20 is preferably provided with an outwardly extending lip 24a, although again such is not required.

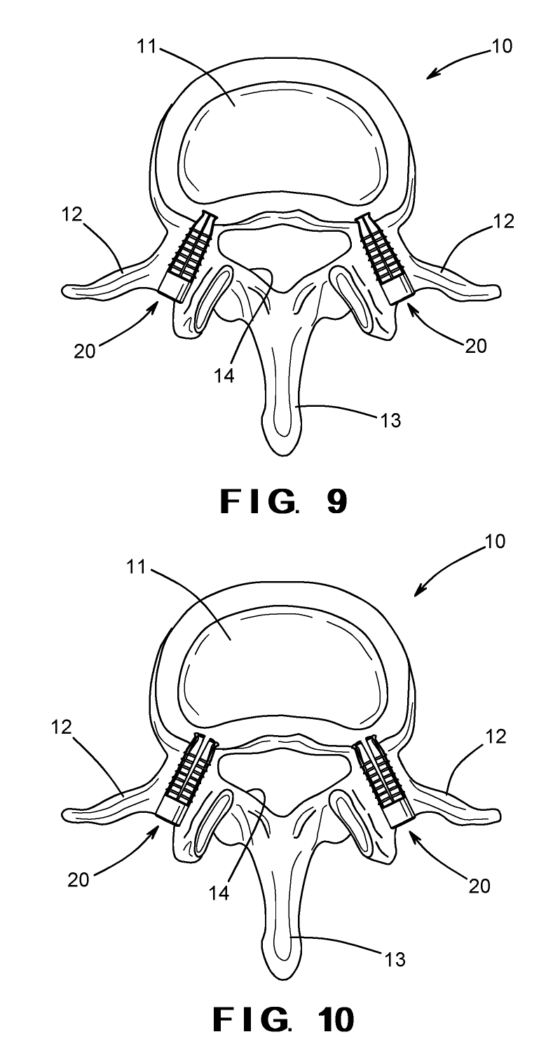

FIGS. 9 and 10 illustrate the vertebra 10 of the spine illustrated in FIG. 2 having two of the first embodiment of the pedicle reconstruction devices 20 provided therein. In FIG. 9, the pedicle reconstruction devices 20 are shown installed in the respective pedicles 12 of the vertebra 10 prior to the installation of the pedicle screws 15 (not shown) therein. Thus, both of the pedicle reconstruction devices 20 are shown in the retracted condition. The pedicle reconstruction devices 20 may, for example, be installed in the respective pedicles 12 of the vertebra 10 by initially drilling or otherwise forming respective openings in such pedicles 12, then axially inserting the pedicle reconstruction devices 20 in such openings (more details regarding this installation procedure is provided below). Thereafter, the pedicle screws 15 (not shown) may be threaded or otherwise inserted respectively within the pedicle reconstruction devices 20. In FIG. 10, the pedicle reconstruction devices 20 are shown installed in the respective pedicles 12 of the vertebra 10 after the installation of the pedicle screws 15 (not shown) therein. Thus, both of the pedicle reconstruction devices 20 are shown in the expanded condition. In this expanded condition, the annular barbs 22a provided on the outer surface of the stem portion 22 and the outwardly extending lip 23a provided on the anchor portion 23 securely engage the adjacent portions of the pedicles 12 of the vertebra 10.

Thus, the pedicle reconstruction device 20 of this invention strengthens the associated pedicle 12 in which it is installed so that the pedicle screw 15 can be inserted safely. In case of medial wall or lateral wall damage of the pedicle 12, the pedicle reconstruction device 20 reconstructs the pedicle 12, making it possible for a surgical construct to be built safely without worry about loosening of the pedicle screw 15, damage to nerve structure, or fracture of the pedicle 12 itself. If desired, the pedicle reconstruction device 20 of this invention may be preformed to have the expanded shape shown in FIG. 8 even prior to the installation of the pedicle screw 15 therein.

The pedicle reconstruction device 20 may formed from any desired material or combination of materials. In particular, the pedicle reconstruction device 20 may be formed from cortical bone, polymeric materials, and metallic materials. The material or combination of materials used to form the pedicle reconstruction device 20 may be either absorbable or not absorbable. The pedicle reconstruction device 20 can be made in such a manner that the walls can carry biomaterials in the pedicle 15 so as to strengthen the pedicle 15 after installation of the pedicle reconstruction device 20 therein.

The pedicle reconstruction device 20 of this invention mitigates the problems associated with pedicle screw 15 loosening in an osteoporotic patient. Nevertheless, it can also be used in patients with normal bone quality to strengthen the anchoring of the pedicle screw 15. There are two important aspects for its proper functioning. As discussed above, the pedicle reconstruction device 20 has four regions along its length, namely, the head portion 21, the stem portion 22, the neck portion 23, and the anchor portion 24. The head portion 21 is, in the illustrated embodiment, the non-expanding region of the pedicle reconstruction device 20. The head portion 21 may have the serrations 21a provided on its inner surface to provide a rough frictional contact (and, therefore, a higher frictional coefficient) with the pedicle screw 15 when inserted therein. It can also provide an additional safety measure because the head portion 21 can be designed to have very low clearance with the head for a tight fit. In other words, this means that a high normal force will come from a tight fit, and a higher coefficient of friction will be an attribute of the serrations 21a. The combined effect of these two factors will result in higher friction. The stem portion 22, the neck portion 23, and the anchor portion 24 of the pedicle reconstruction device 20 may be slotted at regular or other desired intervals to allow these regions to expand when a pedicle screw 15 passes therethrough. The expansion of these three regions results in tight junction fits between both (1) the pedicle screw 15 and the pedicle reconstruction device 20, and (2) the pedicle reconstruction device 20 and the bone of the pedicle 12. The stem portion 22 of the pedicle reconstruction device 20 may have sharp barb-like anchors 22a provided on its outer surface, which can penetrate inside the bone of the pedicle 12 and provide additional anchorage between the bone of the pedicle 12 and pedicle reconstruction device 20. The neck portion 23 of the pedicle reconstruction device 20 may be more tapered than the stem portion 22, which can assist in the gradual expansion of the distal anchor portion 24 inside the inner cortical wall at the junction of the pedicle 12 and vertebral body 11. The anchor portion 24 of the pedicle reconstruction device 20 may have the protruding lip 24a at the outer surface for anchorage against the inner cortical wall of the vertebral body 11. The inner wall of the anchor portion 24 can be increased in thickness (as described in detail below) if further expansion of the outer lip 24a is necessary or desirable. Similarly, the protrusion of the outer lip 24a may be made larger to achieve the same result.

Another advantage of the pedicle reconstruction device 20 of this invention is its bone-like elastic modulus material, which is preferably orders of magnitude lower than metals like titanium. For example, the elastic modulus material may be PEEK, PLA, de-mineralized bone, and the like. These materials have properties that will help in flexibility and in better load transfer between the bone of the pedicle 12 and the pedicle screw 15. This better load transfer will reduce the amount of loosening of the pedicle screw 15 due to fatigue.

An exemplary method for installing the pedicle reconstruction device 20 of this invention in the pedicle 12 will now be described. When revision spine surgery or primary spine surgery is carried out for a damaged or weakened pedicle, the following method can be used to place the pedicle reconstruction device 20. This method can be used either in either minimally invasive spine surgery or open surgery.

Step 1—a guide wire (not shown) is placed within the pedicle 12, preferably using conventional imaging techniques to insure that the guide wire is placed centrally within the pedicle 12 (alternatively, a working cannula can be placed centrally within the pedicle 12 when using minimally invasive surgery techniques).

Step 2—a cannulated drill is inserted over the guide wire, slowly dilating the opening in the pedicle 12 from, for example, about 5 mm to about 12 mm, depending upon the size of the pedicle 12, the size of implant, and the size of the pedicle screw 15 to be installed.

Step 3—the proximal end of the pedicle 12 may be smoothed using a conventional specialized instrument.

Step 4—the pedicle reconstruction device 20 is placed over the guide wire with a specially designed medial reconstruction device implanting device.

Step 5—the guide wire and the implanting device are removed.

Step 6—the pedicle screw 15 is inserted through the pedicle reconstruction device 20 (in the case of a pedicle screw 15 that is cannulated, the pedicle screw 15 could be inserted through the guide wire while it remains in place on the pedicle 12).

Thus, the pedicle reconstruction device 20 of this invention particularly addresses the problem of a damaged or weakened pedicle 12 of the vertebra 10 of the spine, as well as any other boney anatomy of the body. In any spinal surgery where a pedicle screw 15 is inserted, the pedicle 12 plays an important role in the stability of the pedicle screw 15. The pedicle reconstruction device 20 of this invention provides stability to a damaged pedicle 12 and, in the case of medial wall damage, provides protection to the neural structures in the spinal canal by reconstructing the medial wall during revision reconstruction. The pedicle reconstruction device 20 of this invention also provides strength to a weak pedicle 12 containing osteopenia or osteoporotic bone as a preventative measure against loosening of the pedicle screw 15. The pedicle reconstruction device 20 of this invention can also be used in connection with other anatomies having boney structures, such as the hip for example, for stabilization of a screw fixation.

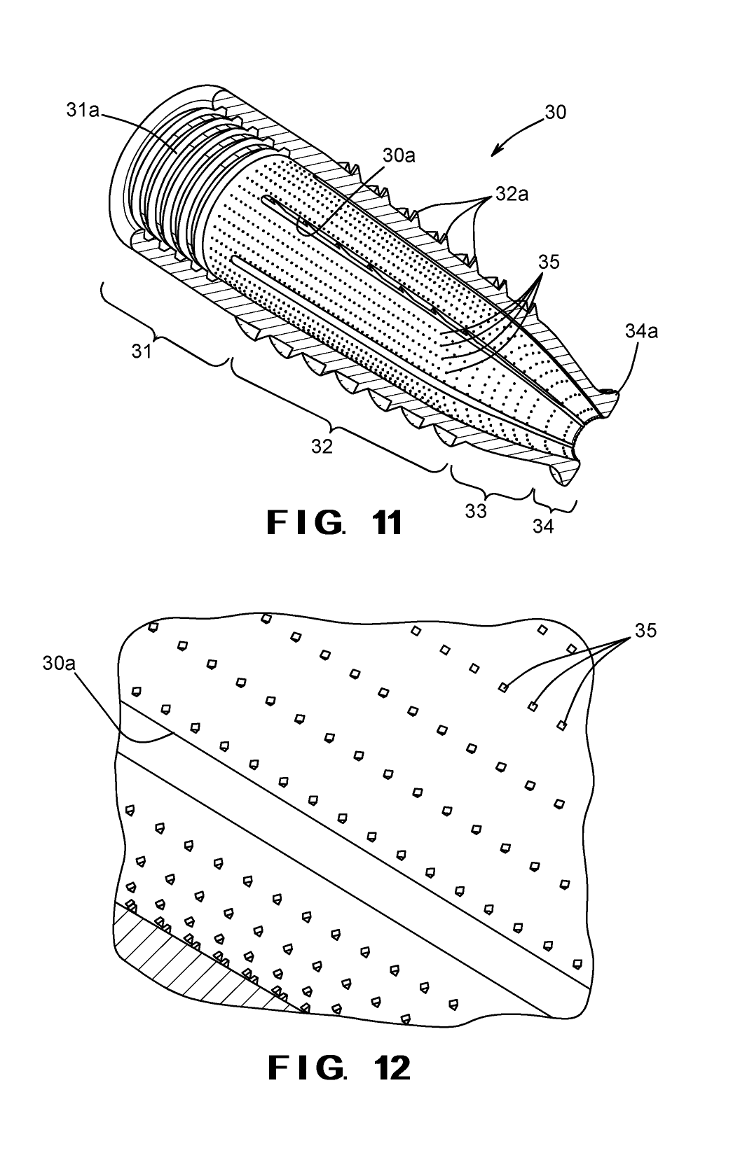

FIGS. 11 and 12 illustrate a second embodiment of a pedicle reconstruction device, indicated generally at 30, in accordance with this invention. The basic structure and manner of operation of the second embodiment of the pedicle reconstruction device 30 is similar to the first embodiment of the pedicle reconstruction device 20 described above, and like reference numbers (incremented by ten) are used to identify similar structures. In this second embodiment of the pedicle reconstruction device 30, the entire inner surface thereof (except for the serrations 31a of the head portion 31) is provided with a plurality of inwardly extending protrusions 35. As with the serrations 31a, the inwardly extending protrusions 35 provide a further rough frictional contact (and, therefore, a higher frictional coefficient) with the pedicle screw 15 when inserted therein. The protrusions 35 may have any desired shape or combination of shapes, and further may be provided in any desired arrangement on the entire inner surface of the second embodiment of the pedicle reconstruction device 30.

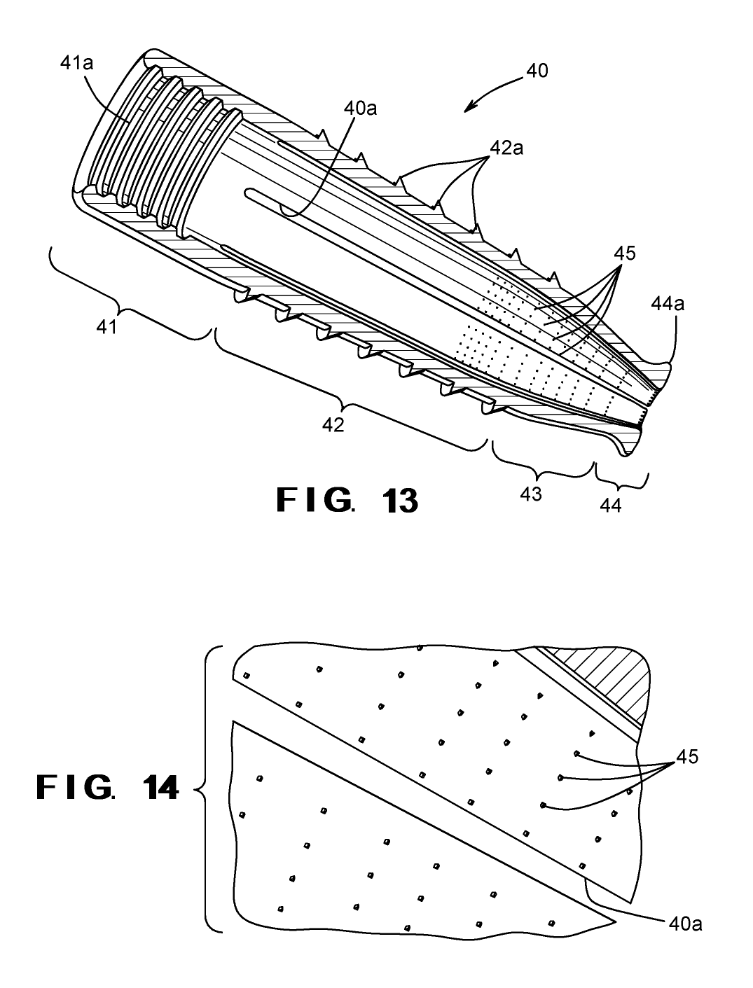

FIGS. 13 and 14 illustrate a third embodiment of a pedicle reconstruction device, indicated generally at 40, in accordance with this invention. The basic structure and manner of operation of the third embodiment of the pedicle reconstruction device 40 is similar to the first embodiment of the pedicle reconstruction device 20 described above, and like reference numbers (incremented by twenty) are used to identify similar structures. In this third embodiment of the pedicle reconstruction device 40, only a portion of the inner surface thereof (except for the serrations 41a of the head portion 41, which are still present) is provided with a plurality of inwardly extending protrusions 45. As with the serrations 41a, the inwardly extending protrusions 45 further provide a rough frictional contact (and, therefore, a higher frictional coefficient) with the pedicle screw 15 when inserted therein. The protrusions 45 may have any desired shape or combination of shapes, and further may be provided in any desired arrangement on the portion of the inner surface of the pedicle reconstruction device 40.

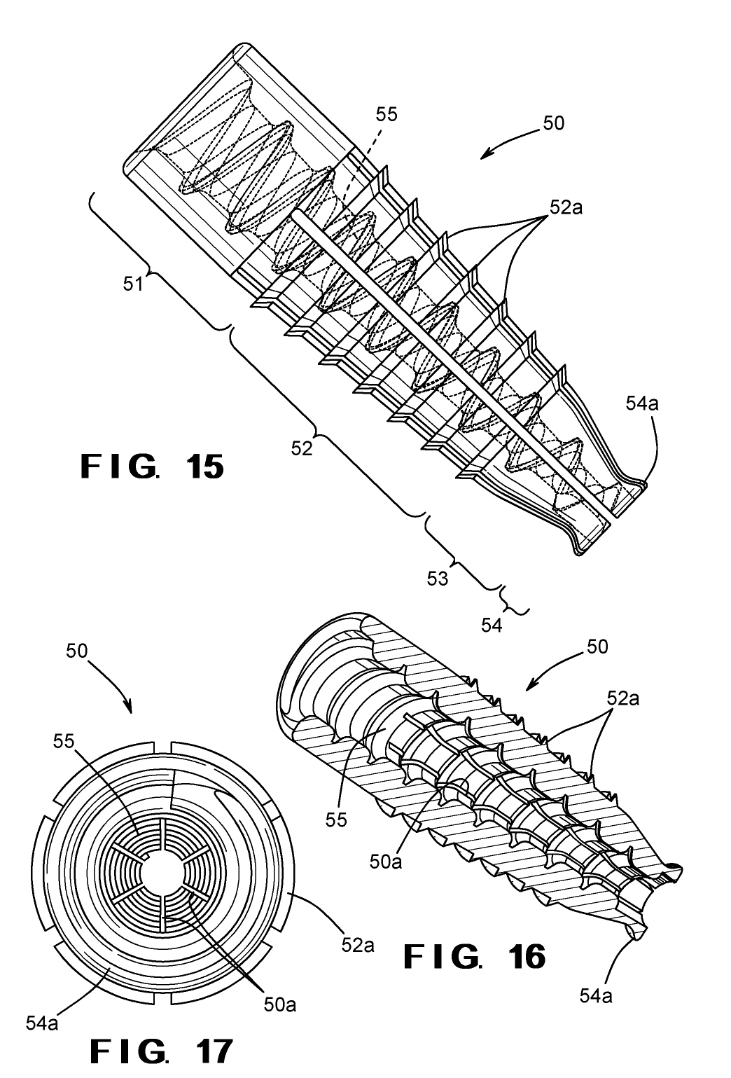

FIGS. 15 through 17 illustrate a fourth embodiment of a pedicle reconstruction device, indicated generally at 50, in accordance with this invention. The basic structure and manner of operation of the fourth embodiment of the pedicle reconstruction device 50 is similar to the first embodiment of the pedicle reconstruction device 20 described above, and like reference numbers (incremented by thirty) are used to identify similar structures. In this fourth embodiment of the pedicle reconstruction device 50, the inner surface thereof is provided with a thread 55 that extends throughout the length thereof. The thread 55 is provided to cooperate with a thread provided on the outer surface of a shank portion of the pedicle screw 15 (not shown) when inserted therethrough, as described above. If desired, the thread 55 may extend throughout only a portion of the length of the fourth embodiment of the pedicle reconstruction device 50.

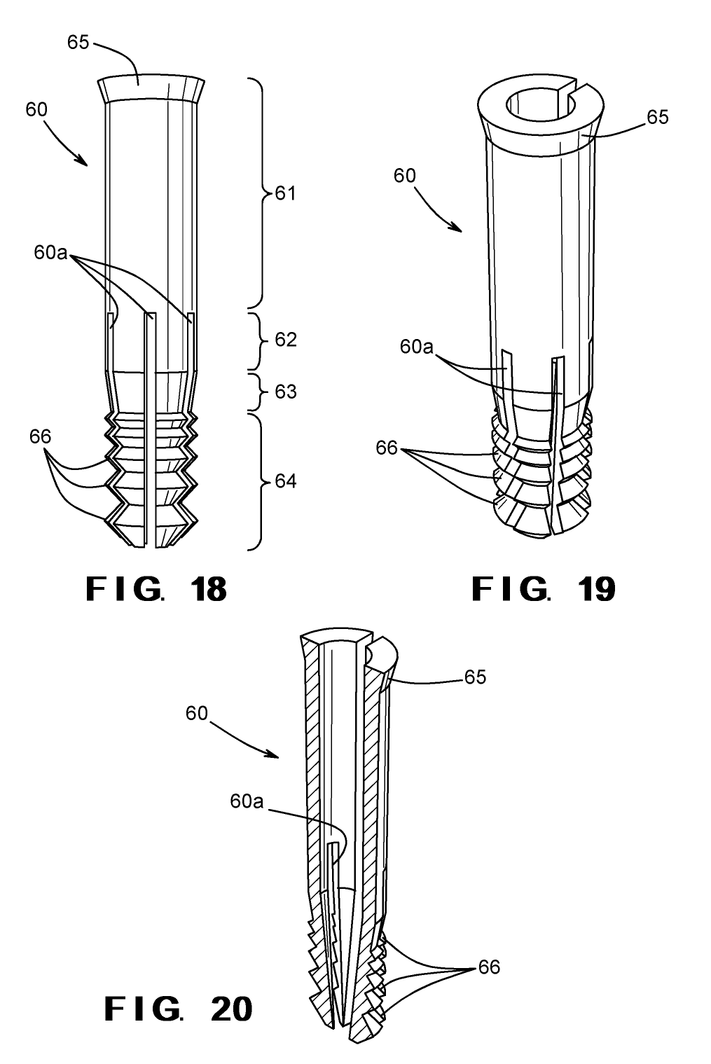

FIGS. 18 through 20 illustrate a fifth embodiment of a pedicle reconstruction device, indicated generally at 60, in accordance with this invention. The basic structure and manner of operation of the fifth embodiment of the pedicle reconstruction device 60 is similar to the first embodiment of the pedicle reconstruction device 20 described above, and like reference numbers (incremented by forty) are used to identify similar structures. In this fifth embodiment of the pedicle reconstruction device 60, the outermost end of the head portion 61 is provided with an outwardly extending lip 65. The lip 65 may function as a positive stop to limit the amount by which the fifth embodiment of the pedicle reconstruction device 60 may be inserted within the pedicle 15, as described above. Also, the anchor portion 64 of the fifth embodiment of the pedicle reconstruction device 60 is provided with a plurality of outwardly extending lips 66, for the same reasons set forth above. Lastly, as best shown in FIG. 20, the wall thickness of the anchor portion 64 of the fifth embodiment of the pedicle reconstruction device 60 gradually increases from the wall thickness of the neck portion 63 to the end of the anchor portion 64. As a result, when the pedicle screw 15 is inserted through the fifth embodiment of the pedicle reconstruction device 60, the anchor portion 64 will be moved even further outwardly in the expanded condition. This provides a more secure engagement of the adjacent portions of the pedicles 12 of the vertebra 10.

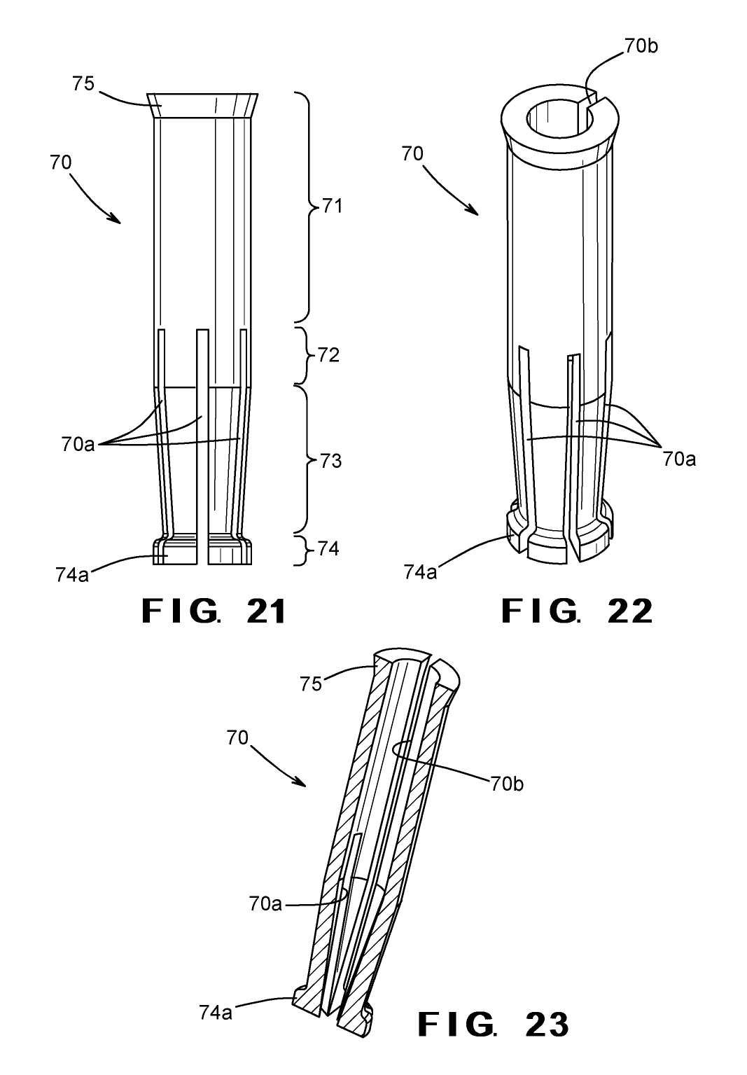

FIGS. 21 through 23 illustrate a sixth embodiment of a pedicle reconstruction device, indicated generally at 70, in accordance with this invention. The basic structure and manner of operation of the sixth embodiment of the pedicle reconstruction device 70 is similar to the first embodiment of the pedicle reconstruction device 20 described above, and like reference numbers (incremented by fifty) are used to identify similar structures. In this sixth embodiment of the pedicle reconstruction device 70, a slot 70b extends completely throughout the axial length thereof. This slot 70b provides the sixth embodiment of the pedicle reconstruction device 70 with an additional amount of flexibility, which may facilitate the insertion thereof into the pedicle 12 of the vertebra 10.

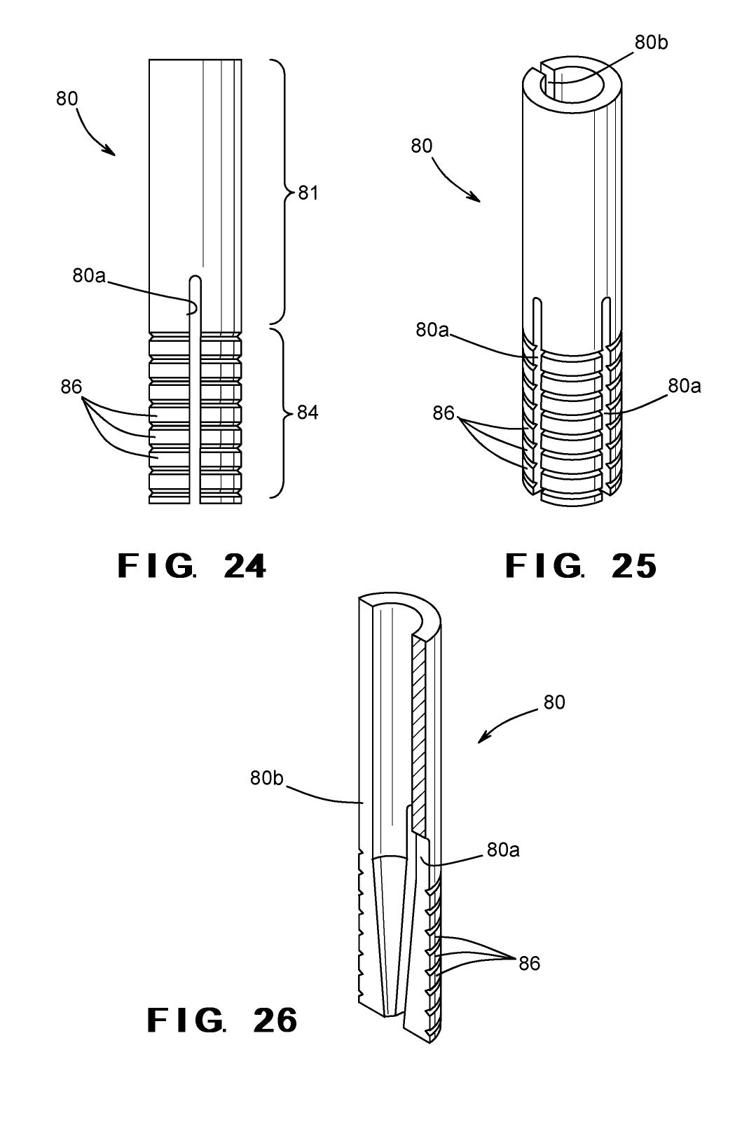

FIGS. 24 through 26 illustrate a seventh embodiment of a pedicle reconstruction device, indicated generally at 80, in accordance with this invention. The basic structure and manner of operation of the seventh embodiment of the pedicle reconstruction device 80 is similar to the first embodiment of the pedicle reconstruction device 20 described above, and like reference numbers (incremented by sixty) are used to identify similar structures. In this seventh embodiment of the pedicle reconstruction device 80, a slot 80b extends completely throughout the axial length thereof, the anchor portion 84 is provided with a plurality of outwardly extending lips 86, and the wall thickness of the anchor portion 84 gradually increases directly from the wall thickness of the head portion 81 to the end of the anchor portion 84, all for the same reasons set forth above.

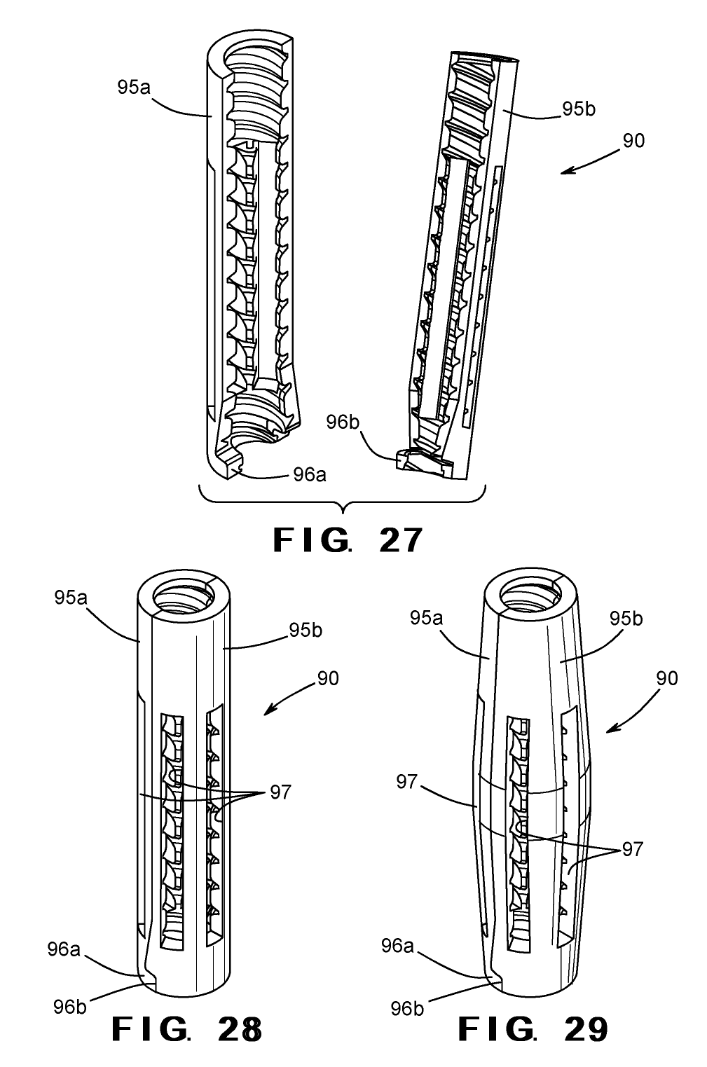

FIGS. 27 through 29 illustrate an eighth embodiment of a pedicle reconstruction device, indicated generally at 90, in accordance with this invention. The basic structure and manner of operation of the eighth embodiment of the pedicle reconstruction device 90 is similar to the first embodiment of the pedicle reconstruction device 20 described above, and like reference numbers (incremented by seventy) are used to identify similar structures. In this eighth embodiment, the pedicle reconstruction device 90 is split into two portions 95a and 95b. If desired, one or more locking structures 96a and 96b may be provided on either or both of the portions 95a and 95b to selectively retain them together for use, as shown in FIGS. 28 and 29. Additionally, the eighth embodiment of the pedicle reconstruction device 90 has a plurality of generally axially extending windows 97 provided therein in lieu of the slots described above. The windows 97 are provided to facilitate the expansion of an internal portion (as opposed to an end portion) of the eighth embodiment of the pedicle reconstruction device 90 from the retracted condition illustrated in FIG. 28 (i.e., prior to the installation of a pedicle screw 15 therein) to the expanded condition illustrated in FIG. 29 (i.e., after the installation of a pedicle screw 15 therein).

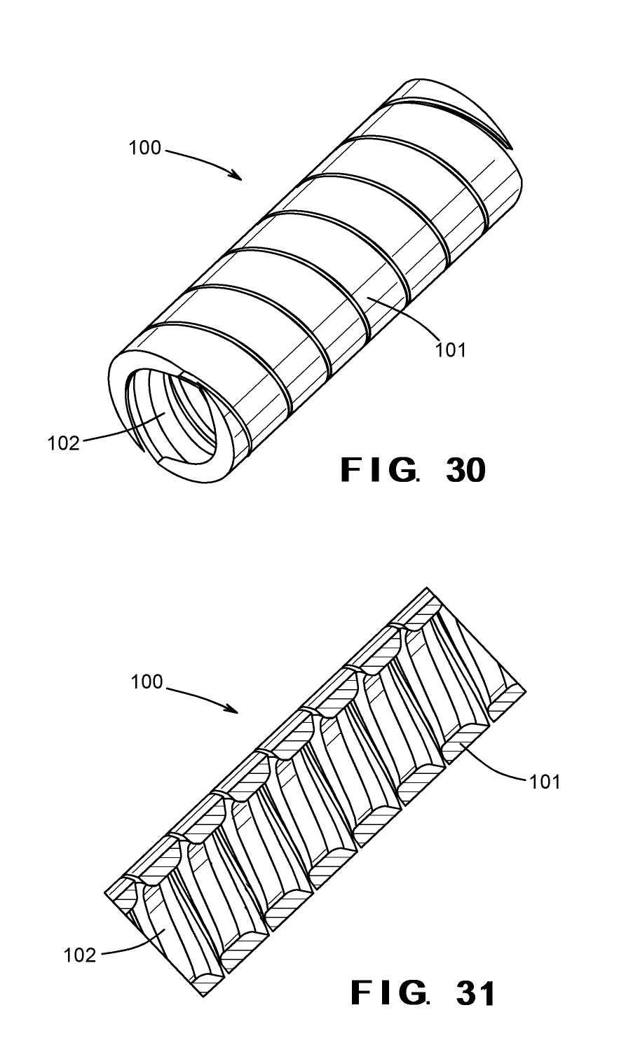

FIGS. 30 and 31 illustrate a ninth embodiment of a pedicle reconstruction device, indicated generally at 100, in accordance with this invention. This ninth embodiment of the pedicle reconstruction device 100 is formed from a single strip of material 101 that is helically shaped to take the overall general form of a hollow cylinder. The outer surface of this ninth embodiment of the pedicle reconstruction device 100 is smooth. If desired, a thread 102 may be provided on the inner surface of the strip of material 101. The thread 102 is provided to cooperate with the thread provided on the outer surface of the shank of the pedicle screw 15 (not shown) when inserted therethrough, as described above. If desired, the thread 102 may extend throughout only a portion of the length of the ninth embodiment of the pedicle reconstruction device 100.

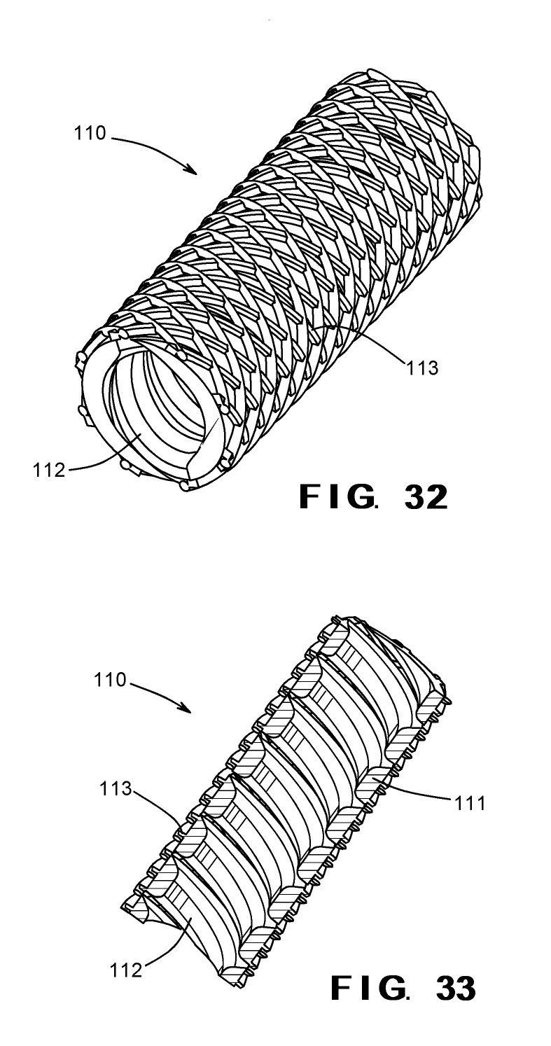

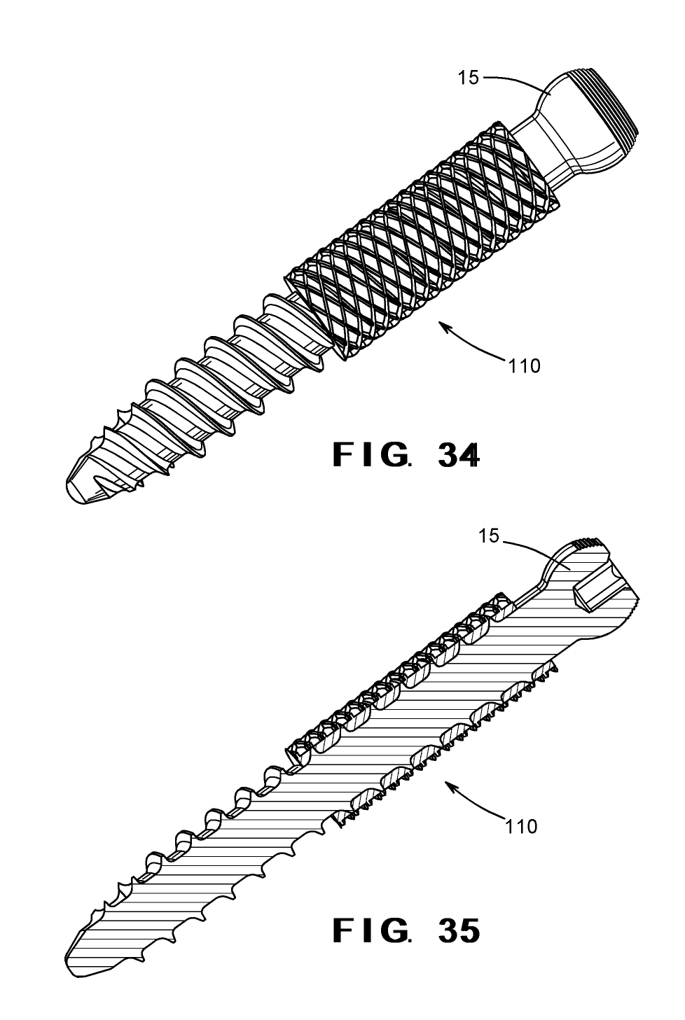

FIGS. 32 through 36 illustrate a tenth embodiment of a pedicle reconstruction device, indicated generally at 110, in accordance with this invention. This tenth embodiment of the pedicle reconstruction device 110 is formed from a single strip of material 111 that is helically shaped to take the overall general form of a hollow cylinder. The outer surface of this tenth embodiment of the pedicle reconstruction device 110 is cross-hatched 113 or is otherwise provided with a non-smooth shape to facilitate the secure engagement thereof with the pedicle 12 of the vertebra 10. If desired, a thread 112 may be provided on the inner surface of the strip of material 111. The thread 112 is provided to cooperate with the thread provided on the outer surface of the shank of the pedicle screw 15 (see FIGS. 34 and 35) when inserted therethrough, as described above. If desired, the thread 112 may extend throughout only a portion of the length of the tenth embodiment of the pedicle reconstruction device 110.

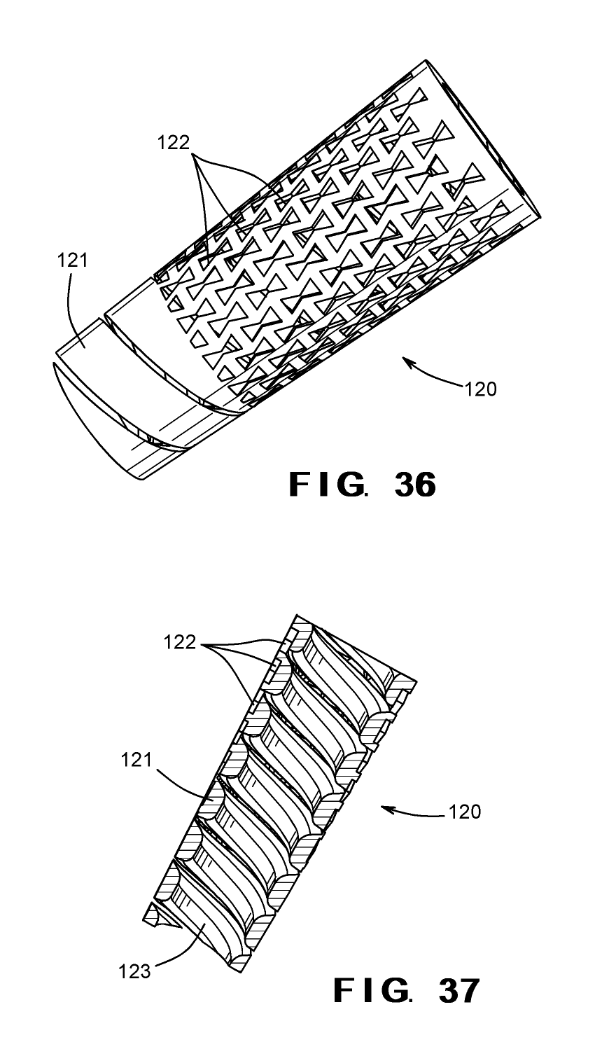

FIGS. 36 and 37 illustrate an eleventh embodiment of a pedicle reconstruction device, indicated generally at 120, in accordance with this invention. This eleventh embodiment of the pedicle reconstruction device 120 is formed from a single strip of material 121 that is helically shaped to take the overall general form of a hollow cylinder. The outer surface of this eleventh embodiment of the pedicle reconstruction device 120 is generally smooth. However, a plurality of openings 122 or recesses are provided in at least a portion of this outer surface to facilitate the secure engagement thereof with the pedicle 12 of the vertebra 10. The illustrated openings 122 are generally hourglass-shaped and are regularly spaced apart from one another. However, the openings 122 or recesses may have any desired shape or combination of shapes, and further may be provided in any desired arrangement on the entire outer surface of the eleventh embodiment of the pedicle reconstruction device 120. If desired, a thread 123 may be provided on the inner surface of the strip of material 121. The thread 123 is provided to cooperate with the thread provided on the outer surface of the shank of the pedicle screw 15 (not shown) when inserted therethrough, as described above. If desired, the thread 123 may extend throughout only a portion of the length of the eleventh embodiment of the pedicle reconstruction device 120.

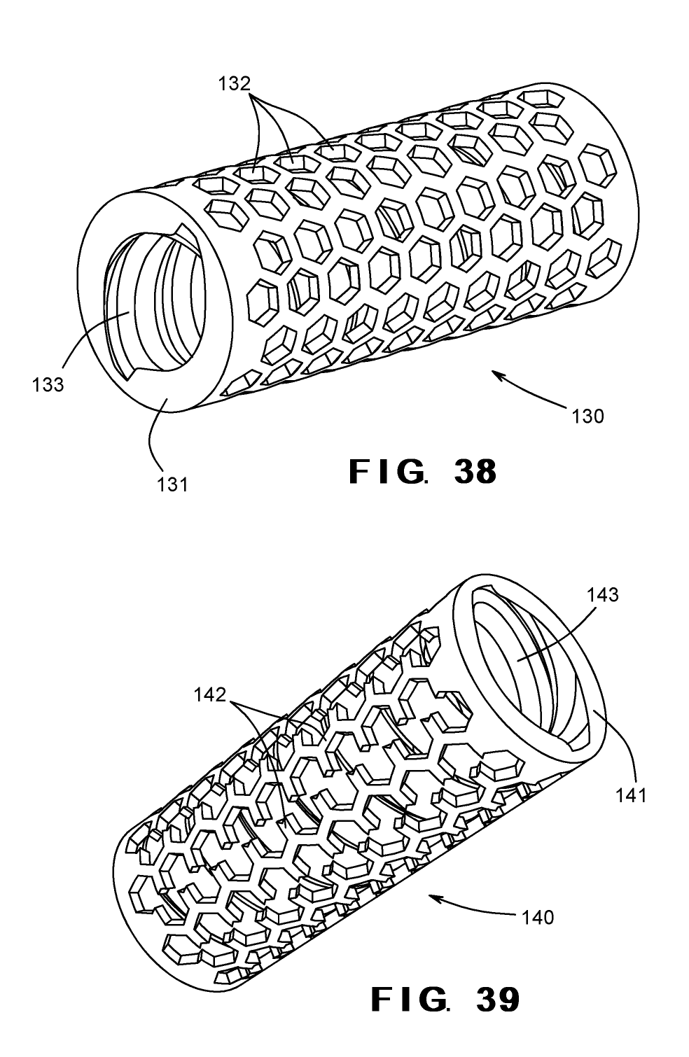

FIG. 38 illustrates a twelfth embodiment of a pedicle reconstruction device, indicated generally at 130, in accordance with this invention. This twelfth embodiment of the pedicle reconstruction device 130 is formed from a single piece of material 131 that is generally hollow and cylindrical in shape. The outer surface of this twelfth embodiment of the pedicle reconstruction device 130 has a plurality of openings 132 or recesses provided through the entire portion thereof to facilitate the secure engagement thereof with the pedicle 12 of the vertebra 10. The illustrated openings 132 are generally hexagonally-shaped and are regularly spaced apart from one another. However, the openings 132 or recesses may have any desired shape or combination of shapes, and further may be provided in any desired arrangement on the outer surface of the twelfth embodiment of the pedicle reconstruction device 130. If desired, a thread 133 may be provided on the inner surface of the single piece of material 131. The thread 133 is provided to cooperate with the thread provided on the outer surface of the shank of the pedicle screw 15 (not shown) when inserted therethrough, as described above. If desired, the thread 133 may extend throughout only a portion of the length of the twelfth embodiment of the pedicle reconstruction device 130.

FIG. 39 illustrates a thirteenth embodiment of a pedicle reconstruction device, indicated generally at 140, in accordance with this invention. This thirteenth embodiment of the pedicle reconstruction device 140 is formed from a single piece of material 141 that is generally hollow and cylindrical in shape. The outer surface of this thirteenth embodiment of the pedicle reconstruction device 140 has a plurality of openings 142 or recesses provided through the entire portion thereof (although such is not required) to facilitate the secure engagement thereof with the pedicle 12 of the vertebra 10. The illustrated openings 142 are generally hexagonally-shaped and are connected to one another throughout the axial length thereof. However, the openings 142 or recesses may have any desired shape or combination of shapes, and further may be provided in any desired arrangement on the outer surface of the thirteenth embodiment of the pedicle reconstruction device 140. If desired, a thread 143 may be provided on the inner surface of the single piece of material 141. The thread 143 is provided to cooperate with the thread provided on the outer surface of the shank of the pedicle screw 15 (not shown) when inserted therethrough, as described above. If desired, the thread 143 may extend throughout only a portion of the length of the thirteenth embodiment of the pedicle reconstruction device 140.

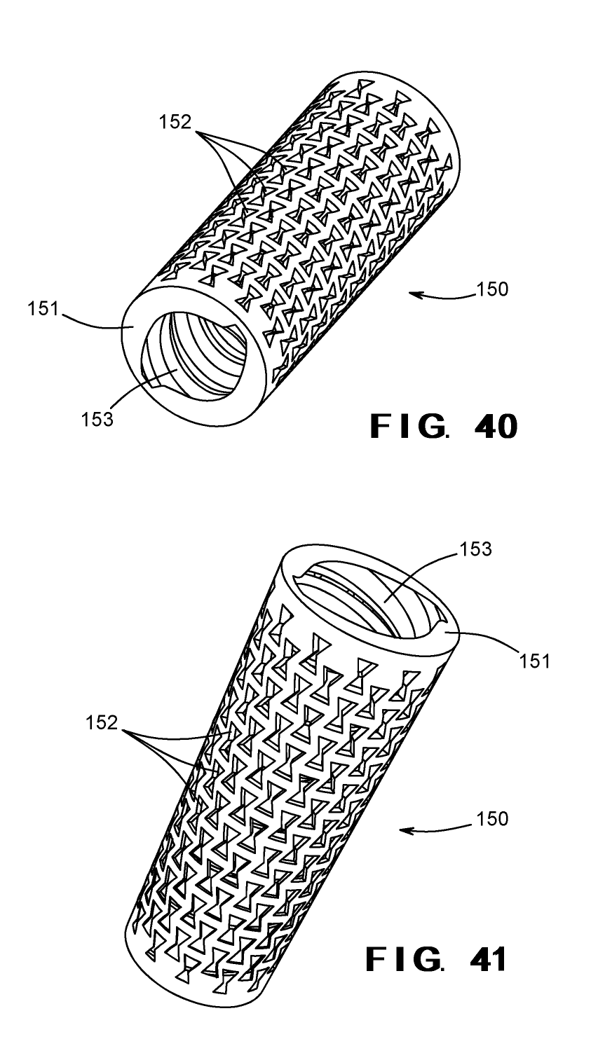

FIGS. 40 and 41 illustrate a fourteenth embodiment of a pedicle reconstruction device, indicated generally at 150, in accordance with this invention. This fourteenth embodiment of the pedicle reconstruction device 150 is formed from a single piece of material 151 that is generally hollow and cylindrical in shape. The outer surface of this fourteenth embodiment of the pedicle reconstruction device 150 is generally smooth. However, a plurality of openings 152 or recesses are provided in a portion of this outer surface to facilitate the secure engagement thereof with the pedicle 12 of the vertebra 10. The illustrated openings 152 are generally hourglass-shaped and are regularly spaced apart from one another. However, the openings 152 or recesses may have any desired shape or combination of shapes, and further may be provided in any desired arrangement on the entire outer surface of the fourteenth embodiment of the pedicle reconstruction device 150. If desired, a thread 153 may be provided on the inner surface of the single piece of material 151. The thread 153 is provided to cooperate with the thread provided on the outer surface of the shank of the pedicle screw 15 (not shown) when inserted therethrough, as described above. If desired, the thread 153 may extend throughout only a portion of the length of the fourteenth embodiment of the pedicle reconstruction device 150.

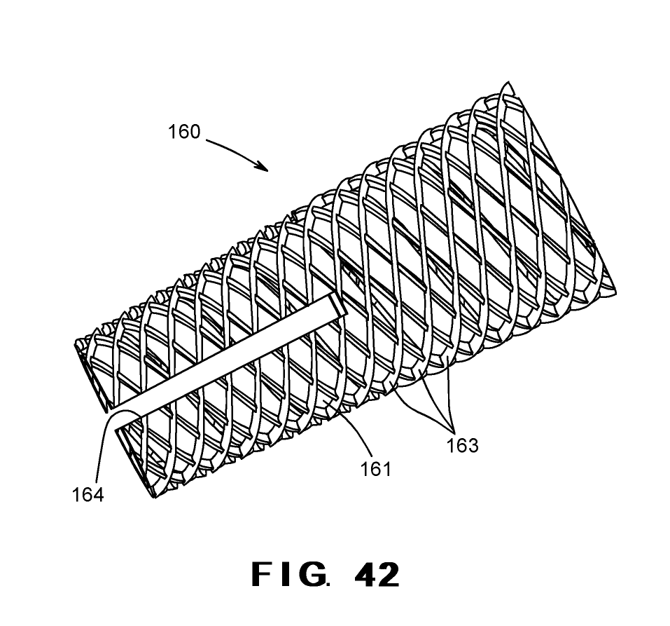

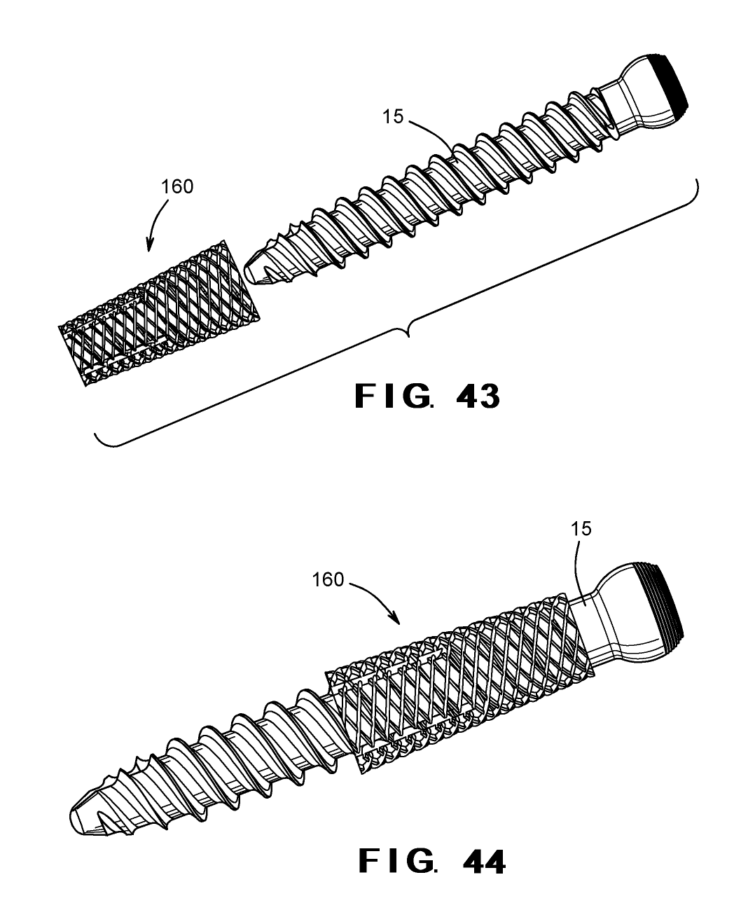

FIGS. 42 through 44 illustrate a fifteenth embodiment of a pedicle reconstruction device, indicated generally at 160, in accordance with this invention. This fifteenth embodiment of the pedicle reconstruction device 160 is formed from a single piece of material 161 that is generally hollow and cylindrical in shape. The outer surface of this fifteenth embodiment of the pedicle reconstruction device 160 is cross-hatched 163 or is otherwise provided with a non-smooth shape to facilitate the secure engagement thereof with the pedicle 12 of the vertebra 10. If desired, a thread (not shown) may be provided on the inner surface of the single piece of material 161. The thread is provided to cooperate with the thread provided on the outer surface of the shank of the pedicle screw 15 (see FIGS. 43 and 44) when inserted therethrough, as described above. If desired, the thread 162 may extend throughout only a portion of the length of the fifteenth embodiment of the pedicle reconstruction device 160. One or more slots 163 may be formed throughout portions of the single piece of material 161.

The principle and mode of operation of this invention have been explained and illustrated in its preferred embodiments. However, it must be understood that this invention may be practiced otherwise than as specifically explained and illustrated without departing from its spirit or scope.

What is claimed is:

1. A pedicle reconstruction device adapted for use with a pedicle screw, the pedicle reconstruction device comprising:
a head portion;
a stem portion extending from the head portion;
a neck portion extending from the stem portion; and
an anchor portion extending from the neck portion,
wherein a slot is formed through the neck portion and the anchor portion such that the anchor portion can be oriented in a retracted condition when pedicle screw is not disposed therein and in an extended condition when a pedicle screw is disposed therein.

2. The pedicle reconstruction device defined in claim 1 wherein the head portion includes an inner surface that is non-smooth in shape.

3. The pedicle reconstruction device defined in claim 1 wherein the stem portion includes an outer surface that is non-smooth in shape.

4. The pedicle reconstruction device defined in claim 3 wherein the outer surface of the stem portion has a plurality of annular barbs provided thereon.

5. The pedicle reconstruction device defined in claim 1 wherein the stem portion includes an inner surface that is non-smooth in shape.

6. The pedicle reconstruction device defined in claim 5 wherein the inner surface of the stem portion has a plurality of inwardly extending protrusions provided thereon.

7. The pedicle reconstruction device defined in claim 1 wherein the neck portion tapers from a relatively large size adjacent the stem portion down to relatively small size adjacent the anchor portion.

8. The pedicle reconstruction device defined in claim 1 wherein the anchor portion has an outwardly extending lip provided thereon.

9. The pedicle reconstruction device defined in claim 8 wherein the anchor portion has a plurality of outwardly extending lips provided thereon.

10. The pedicle reconstruction device defined in claim 1 wherein a plurality of slots is formed through the neck portion and the anchor portion.

11. The pedicle reconstruction device defined in claim 10 wherein the slot is formed completely through the head portion, the stem portion, the neck portion. and the anchor portion.

12. The pedicle reconstruction device defined in claim 1 wherein a thread is provided on inner surfaces of the neck portion and the anchor portion.

13. The pedicle reconstruction device defined in claim 12 wherein the thread is provided on inner surfaces of the head portion, the stem portion, the neck portion. and the anchor portion.

14. The pedicle reconstruction device defined in claim 1 wherein the head portion is provided with an outwardly extending lip.

15. The pedicle reconstruction device defined in claim 1 wherein the head portion, the stem portion, the neck portion. and the anchor portion are is split into two portions.

16. The pedicle reconstruction device defined in claim 15 wherein one or more locking structures are provided on either or both of the portions to selectively retain them together for use.
